(19) United States
(12) Patent Application Publication
(10) Pub. No.: US 2003/0171735 A1
(43) Pub. Date: Sep. 11, 2003

(54) Charging hypodermic syringes with injectant

(76) Inventors: David William Parker, Lancashire (GB); Colin Hamilton Burgess, Lancashire (GB)

Correspondence Address: NIXON & VANDERHYE, PC, 1100 N GLEBE ROAD, 8TH FLOOR, ARLINGTON, VA, 22201-4714, US

(21) Appl. No.: 10/257,922

(22) PCT Filed: Jul. 25, 2001
(86) PCT No.: PCT/GB01/01854

(30) Foreign Application Priority Data
Apr. 27, 2000 (GB) 0010307.7

(51) Int. Cl. A61M 5/32

(52) U.S. Cl. 604/507; 128/898; 604/414

(57) ABSTRACT

A hypodermic syringe (1, 2) includes a needle (3) covered by a needle sheath (4) that comprises a tubular sheath body (5) with a removable end cap (6). Injectant is transferred into the main body (1) of the syringe by removing the end cap, inserting the needle together with the sheath body (5) into a source of injectant and operating the plunger so as to draw the injectant through open end (6) of the plunger through the needle into the main body. The process is carried out without exposing the needle to the user's touch.

### FIELD OF THE INVENTION

This invention relates to charging hypodermic syringes with injectant and has particular but not exclusive application to hypodermic syringes which have a needle covered by a needle sheath to prevent needle stick injuries.

### BACKGROUND

Conventionally, hypodermic syringes are charged with injectant by placing the needle of the syringe into a reservoir of injectant and operating the syringe's plunger to cause induction of the injectant into the main body of the syringe through the needle. The needle may be provided with a protective sheath which is removed in order to allow the needle to take up the injectant during the charging process. However, a problem with this prior technique is that there is a risk that the user may suffer a needle stick injury from the exposed needle, in which the user's skin becomes punctured by the needle.

The risk of needle stick injuries during the charging of hypodermic syringes has given rise to strict and costly regulations and procedures, particularly with regard to highly toxic and other controlled injectants.

The present invention seeks to provide an improved method of charging the syringe in which the risk of needle stick injury is materially reduced.

### SUMMARY OF THE INVENTION

According to the invention, there is provided a method of charging a hypodermic syringe with an injectant, the syringe including a needle covered by a needle sheath, the method comprising opening the needle sheath, and causing induction of an injectant into the syringe through the needle without exposing the needle to the user's touch.

The needle sheath may include a tubular sheath body and a removable end cap and the method may include removing the end cap from the sheath body to allow the induction of injectant through the needle.

The tubular sheath body may have an open end covered by the end cap, and the method may include inserting the open end with the end cap removed, into a reservoir of injectant liquid, and causing the induction of the injectant into the syringe through the open end and into the needle. Alternatively, the tubular sheath body may have a domed end and transverse holes covered by the end cap, through which the injectant may pass.

The needle sheath may be inserted into a dispenser containing the injectant, which may comprise a bulb with a nose piece which can be punctured by the needle. The bulb may contain a gelatinous material.

Alternatively, the dispenser may comprise a container with a removable lid, into which the sheath body may be inserted for induction of the injectant.

The syringe may comprise a syringe body with a plunger slidably received therein, so that induction of the injectant is carried out by operating the plunger.

The syringe may be configured so that the needle is automatically withdrawn into the syringe after its use to inject the injectant, for example into a patient.

The invention also includes a kit of parts for performance of the aforesaid method, comprising a hypodermic syringe including a needle covered by a needle sheath with a removable end cap, and an injectant dispenser containing the injectant, configured so that by removing the end cap the needle can be arranged so that the injectant can be drawn from the dispenser into the syringe through the needle without exposing the needle to the user's touch.

### BRIEF DESCRIPTION OF THE DRAWINGS

In order that the invention may be more fully understood an embodiment thereof will now be described by way of example with reference to the accompanying drawings in which:

FIG. 1 is a schematic partial sectional view of a hypodermic syringe fitted with a needle sheath having a removable end cap;

FIG. 2 is a similar view to FIG. 1 illustrating an alternative end shape for the needle sheath;

FIG. 3 illustrates the hypodermic syringe in combination with an injectant dispenser; and

FIG. 4 illustrates the syringe together with an alternative form of injectant dispenser in the form of a bulb.

### DETAILED DESCRIPTION

Referring to FIG. 1, a hypodermic syringe is shown which comprises a generally cylindrical main body 1 with a plunger 2 and hypodermic needle 3. The syringe may be of any suitable conventional design and, as well known in the art, injectant can be induced into the main body 1 by depression of the plunger 2 and then pulling the plunger outwardly with the needle dipped into injectant liquid so that the injectant is induced into the main body 1 by the outward movement of the plunger 2 through the needle 3.

In order to reduce the risk of needle stick injury, the syringe may be configured so that the needle is automatically withdrawn into the main body 1 after use, as described e.g. in our co-pending application WO 00/18454. With this arrangement, a pre-sheathed needle is fitted into the main body 1 through an aperture from within, thereby reducing the risk of needle injuries.

As shown in FIG. 1, the needle 3 is covered by a needle sheath 4 that comprises a tubular sheath body 5 with an open end 6 that is covered by an end cap 7 on an integral flexible connection 8 coupled to the sheath body 5. The end cap 7 is shown removed from the sheath body 5, ready to receive the injectant as will be described later. The end cap 7 can be positioned on the end of the sheath body 5 as shown in hatched outline.

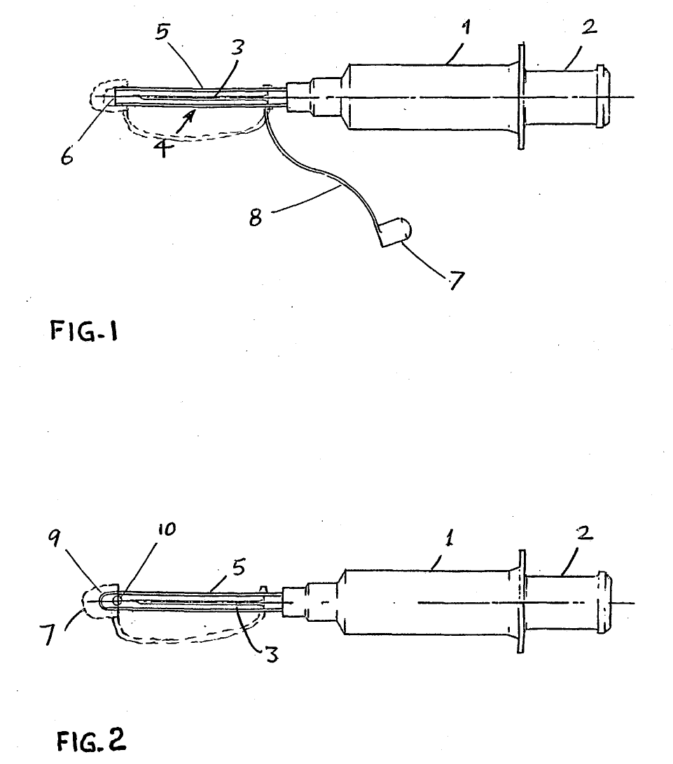

FIG. 2 illustrates an alternative version of the needle sheath in which the main body 5 is provided with a domed end 9 and at least one transverse hole 10 covered by the end cap 6.

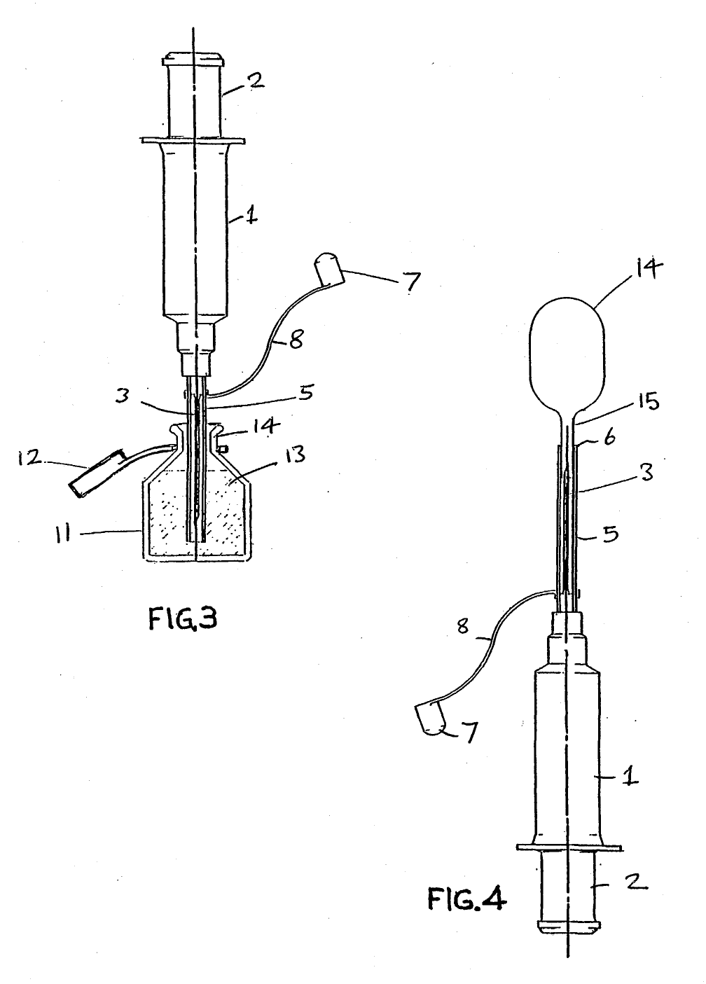

FIG. 3 illustrates how injectant can be induced into the main body of the syringe through the needle from an injectant dispenser 11 in the form of a container with a removable lid 12, filled with injectant liquid 13. In use, the end cap 7 is removed and the needle sheath body 5 is inserted through opening 14 into the container 11. The plunger 2 is then operated to draw injectant liquid through open end 6 of the sheath body 5, through needle 3 into the main body 1 of the syringe. It will be understood that during this process, the needle remains sheathed so that the risk of a needle stick injury is materially reduced. It will be understood that the alternative configuration of needle sheath shown in FIG. 2 can also be used, in which case the injectant is drawn through the transverse holes 10 and then through the needle 3 into the main body 1 of the syringe.

FIG. 4 illustrates an alternative form of container for the injectant, which comprises a bulb 14. The bulb may contain a gelatinous material (not shown) together with the injectant. The bulb 14 has a nose piece 15 which can be fitted into the open end 6 of the tubular sheath body 5. In use, the nose piece 15 becomes punctured by the needle 3, so as to provide access to the injectant within bulb 14. Then, by operating the plunger 2, the injectant can be drawn into the main body 1 of the syringe so as to charge it with the injectant. The tubular sheath body 5 protects the needle 3, so that it is not exposed to the user's touch during the injection induction process.

The charged syringe can then be withdrawn from the container 11 or bulb 14 and the needle sheath cap 7 replaced. When it is desired to use the syringe, the needle sheath 4 is removed in its entirety so as to expose the needle 3 ready for use. Thus, the needle can remain protected during the injection process until the moment at which the syringe is to be used e.g. in a patient. After use, the needle may be automatically retracted within the main body 1 as described in our previously mentioned PCT Application.

1. A method of charging an hypodermic syringe with an injectant, the syringe including a needle covered by a needle sheath, the method comprising opening the needle sheath, and causing induction of an injectant into the syringe through the needle without exposing the needle to the user's touch.

2. A method according to claim 1 wherein the needle sheath includes a tubular sheath body and a removable end cap, and the method includes removing the end cap from the sheath body to allow the induction of injectant through the needle.

3. A method according to claim 2 wherein the tubular sheath body has an open end covered by the removable end cap, and the method includes inserting the open end, with the end cap removed, into a reservoir of injectant liquid, and causing the induction of the injectant into the syringe through the open end and into the needle.

4. A method according to claim 2 wherein the tubular sheath body has a domed end and transverse holes covered by the end cap, and the method includes inserting the domed end, with the end cap removed into a reservoir of injectant liquid, and causing the induction of the injectant into the syringe through the transverse holes and through the needle.

5. A method according to claim 2, 3 or 4 wherein the cap is connected to the main body by a flexible connection, and including removing the cap such that it remains connected to the sheath body by said flexible connection.

6. A method according to any preceding claim including inserting the needle sheath into an injectant dispenser to allow the induction of the injectant from the dispenser into the syringe through the needle without exposing the needle to the user's touch.

7. A method according to claim 6 including puncturing the injectant dispenser with the needle to allow the induction of the injectant.

8. A method according to claim 7 wherein the injectant dispenser comprises a bulb with a nose piece, and including inserting the nose piece into the sheath body to be punctured by the needle.

9. A method according to claim 8 wherein the bulb contains a gelatinous material such that the injectant is released for induction through the needle.

10. A method according to claim 7 wherein the injectant dispenser comprises a container with a removable lid, and the method comprises removing the lid and inserting the sheath body into the container.

11. A method according to any preceding claim wherein the syringe comprises a syringe body and a plunger slidably received in the syringe body, and the induction of the injectant is carried out by operating the plunger.

12. A method according to any preceding claim including automatically withdrawing the needle into the syringe after use thereof to inject the injectant.

13. A kit of parts for performance of the method claimed in claim 1 comprising an hypodermic syringe including a needle covered by a needle sheath with a removable end cap, and an injectant dispenser containing an injectant, configured so that by removing the end cap the needle sheath can be arranged so that the injectant can be drawn from the dispenser into the syringe through the needle without exposing the needle to the user's touch.
